(19) United States
(12) Patent Application Publication
(10) Pub. No.: US 2003/0125714 A1
(43) Pub. Date: Jul. 3, 2003

(54) Dosage form for time-varying patterns of drug delivery

(76) Inventors: David Emil Edgren, Los Altos, CA (US); Patrick S.-L. Wong, Burlingame, CA (US); Shu Li, Union City, CA (US)

Correspondence Address: ALZA CORPORATION, P O BOX 7210, INTELLECTUAL PROPERTY DEPARTMENT, MOUNTAIN VIEW, CA, 940397210

(21) Appl. No.: 10/324,172

(22) Filed: Dec. 18, 2002

Related U.S. Application Data
(60) Provisional application No. 60/341,618, filed on Dec. 18, 2001.

(51) Int. Cl. A61K 9/22

(52) U.S. Cl. 604/892.1; 424/468

(57) ABSTRACT

The present invention provides a multi-release oral drug delivery system that initiates drug release following an initial drug-free release interval, after administration to a subject, and a second drug-free release period before release of another dose of drug. The system has (1) inner compartments enclosed within a semipermeable membrane, and (2) a drug coating on the exterior of the semipermeable membrane surrounded by a microporous membrane, which microporous membrane is permeable to fluid and drug. The drug coating is released after the initial drug-free release interval. An inner compartment drug is released after a second drug-free release interval provided by a drug-free inner compartment.

### FIELD OF THE INVENTION

The present invention relates to a novel dosage form for time-varying patterns of drug delivery. More particularly, the invention relates to a dosage form provided as an osmotic drug delivery device for controlled delivery of a drug in a time-varying pattern.

### BACKGROUND OF THE INVENTION

Presently, pharmacy and medicine provide delivery systems for the constant-rate delivery of a drug to a drug-recipient user. For example, infusion pumps are described in U.S. Pat. No. 4,318,400, oral matrix systems are described in U.S. Pat. No. 4,863,744, osmotic systems are described in U.S. Pat. Nos. 3,845,770 and 3,916,899, osmopolymer-powered systems are provided in U.S. Pat. No. 4,783,337, and implants are discussed in U.S. Pat. No. 4,093,709, for constant-rate delivery of a drug.

More recently, U.S. Pat. Nos. 5,156,850 and 5,232,701 describe drug delivery systems that provide an immediate release dose of a therapeutic drug followed by a drug-free interval followed by a delivery of a dose of the drug. These systems include a dosage form having a body that contains three interior layers and provide a substantially drug-free interval before the dosage form begins delivering the drug. The three interior compartments include: (1) a drug-free compartment for producing a drug-free interval prior to the administration of the drug; (2) a drug compartment; (3) and an expandable compartment that provides the force that pushes the other two layers through an exit orifice. The exit orifice is typically formed adjacent to the drug-free compartment. The dosage form also contains an immediate release drug layer on the exterior of the body.

Other delivery systems which incorporate a delay and some form of pulse delivery of drug utilize an optional exterior coating of drug for immediate release after administration to a patient. Examples of this type of delivery system are in U.S. Pat. Nos. 5,785,994 and 5,156,850

Other systems are designed to provide immediate or controlled release dosages after delays. Examples are U.S. Pat. Nos. 4,498,255 and 5,312,388 and 5,223,265, which utilize a single telescoping gelatin capsule that opens after a delay to release its beneficial agent. U.S. Pat. No. 5,443,459 utilizes a series of nested telescoping gelatin capsules that open and instantly release their beneficial agent in a sequential fashion. U.S. Pat. Nos. 4,786,500 and 4,927,632 utilize a reservoir that bursts after delay. These systems are costly to manufacture and not preferred. U.S. Pat Nos. 4,986,987; 4,948,592; 4,842,867 and 5,200,196 utilize an immediate release coating of drug on the exterior and a delay layer around the remainder of the dosage form to provide the delay before delivery of the remaining beneficial agent. This type of system requires the addition of another layer, the delay layer, in the manufacturing process that adds to the cost and complexity of the system.

Another type of system that incorporates a delay in the delivery of beneficial agent after administration of the dosage form utilizes a modulating agent mixed within the drug composition that allows for a zero order release rate of agent with an initial, intermediate or final pulse release of beneficial agent. Examples of this type of system include U.S. Pat. Nos. 4,751,070; 4,851,229 and 4,777,049. These systems only allow a pulse and zero order release rates and do not allow for a delay or two pulse delivery.

Still others use a dispenser that dispenses a series of discrete solid dosage units. See U.S. Pat. Nos. 5,340,590; 5,023,088; 5,017,381 as examples of this type of system.

There are also enterically coated systems which do not release until there is an increase in the pH of the environment, at which time the enteric coating dissolves and releases the beneficial agent. U.S. Pat. No. 4,851,231 exemplifies this system which is not pH independent allowing for release after a desired period of time, but rather only releases after a change in pH.

While the above-mentioned delivery systems may provide acceptable therapy, there are therapeutic programs that prefer the dose of drug be administered in a time-varying pattern following an optional initial drug-free interval. For example, it is often desirable to administer a dosage form to a patient's prior to retiring and deliver the drug in two pulses after a drug-free interval during sleep. However, no dosage form is currently available that fulfills this unmet need. For instance, manufacturing a two-pulse system having a design analogous to that described in U.S. Pat. Nos. 5,156,850 and 5,232,701 would be impractical. Specifically, fabrication of a delivery system containing five interior compartments namely: (1) a drug-free layer; (2) a drug layer; (3) a second drug-free layer; (4) a second drug layer; and (5) an expandable layer, would require a five-layer tablet press. The manufacture of five-layer tablets requiring precise weight control of the individual layers would pose significant fabrication obstacles as well as significant additional manufacturing costs.

### SUMMARY OF THE INVENTION

The present invention in one embodiment is directed to a two-release drug delivery system that is capable of initiating the administration of a drug following an initial drug-free interval and that does not require a five-layer tablet press for manufacture. The invention is based, in part, on the recognition that an initial drug-free interval followed by a first pulse, of the two-pulse drug delivery system, can be implemented by creating a drug coating with a microporous membrane on a body that contains three interior compartments: (1) a drug-free compartment, (2) a drug compartment, and (3) a push compartment that expands upon exposure to a fluid. The drug delivery system provides good drug rate control and is capable of delivering drug over a duration of about 1 hour to about 3 hours or more.

Alternatively, drug delivery from each release can be a controlled release over an extended period of time of 3 hours to 10 hours or more rather than simple pulse delivery over 1 hour to 3 hours.

Accordingly, in one aspect, the invention is directed to a dosage form for administering doses of drugs once or twice a day in an environment of use from a single dosage form, which dosage form includes:

(a) a compartment;

(b) a semipermeable wall that surrounds and forms the compartment, said semipermeable wall containing a composition that is permeable to the passage of fluid and is substantially impervious to the passage of a drug;

(c) an exterior drug layer containing a dose amount of a first drug on the exterior surface of the semipermeable wall;

(d) microporous membrane coated around the exterior drug layer said microporous membrane having a composition that is permeable to the passage of fluid and is permeable to the passage of the first drug;

(e) a first composition in the compartment, said first composition being drug-free for producing a drug-free interval prior to the administration of drug from the compartment;

(f) a second composition in the compartment containing a dose amount of a second drug for producing a therapeutic effect;

(g) a third composition in the compartment that expands in the presence of fluid that enters the device; and

(h) exit means in the semipermeable wall, external drug layer, and microporous membrane for connecting the exterior of the dosage form with the compartment.

In another aspect, the invention is directed to a method for administering doses of drugs once or twice a day from a single dosage form to the gastrointestinal tract of a warm-blooded animal, which method includes:

(a) admitting the dosage form into the gastrointestinal tract of the warm-blooded animal, said dosage form including:

(1) a compartment;

(2) a semipermeable membrane that surrounds and forms the compartment, said semipermeable membrane comprising a composition that is permeable to the passage of fluid and is substantially impervious to the passage of a drug;

(3) an exterior drug layer containing a dose amount of a first drug on the exterior surface of the semipermeable membrane;

(4) microporous membrane coated around the exterior drug layer said microporous membrane having a composition that is permeable to the passage of fluid and is permeable to the passage of the first drug;

(5) a first composition in the compartment, said first composition being drug-free for producing a drug-free interval prior to the administration of drug from the compartment;

(6) a second composition in the compartment containing a dose amount of a second drug for producing a therapeutic effect;

(7) a third composition in the compartment that expands in the presence of fluid that enters the device; and

(8) exit means in the semipermeable membrane, external drug layer, and microporous membrane for connecting the exterior of the dosage form with the compartment;

(b) administering a dose of the first drug from the exterior drug layer by contacting the dosage form with gastrointestinal fluid;

(c) imbibing gastrointestinal fluid into the compartment thereby causing the third composition to expand and push against the second compartment; and

(d) administering the dose of the second drug from the second composition after the first drug-free composition is released from the compartment.

The present invention in a further embodiment is directed to a three-release drug delivery system that is capable of initially delivering an immediate release first drug coating and then initiating the release of a second drug following a first drug-free interval, that does not require a five-layer tablet press for manufacture, and then initiating the release of a third drug following a second drug-free interval. The first drug, second drug and third drug can be the same or different therapeutic agents. The invention is based, in part, on the recognition that a first drug-free interval followed by a first pulse, of the second drug, can be implemented by creating a drug coating with a microporous membrane on a body that contains three interior compartments: (1) a drug-free compartment, (2) a drug compartment, and (3) a push compartment that expands upon exposure to a fluid. The drug delivery system provides good drug rate control and is capable of delivering drug over a duration of about 1 hour to about 3 hours or more from the second and third drug release.

Alternatively, as above with two-drug delivery, drug delivery from the second and third drug release can be a controlled release over an extended period of time of 3 hours to 10 hours or more rather than pulse delivery over 1 hour to 3 hours. The initial delivery from the immediate release coating would be an immediate release of less than about 1 hour.

Accordingly, in one aspect, the invention is directed to a dosage form for administering doses of drugs once, twice or three times a day in an environment of use from a single dosage form, which dosage form includes:

(a) a compartment;

(b) a semipermeable membrane that surrounds and forms the compartment, said semipermeable membrane containing a composition that is permeable to the passage of fluid and is substantially impervious to the passage of a drug;

(c) an exterior drug layer containing a dose amount of a first drug on the exterior surface of the semipermeable membrane;

(d) microporous membrane coated around the exterior drug layer said microporous membrane having a composition that is permeable to the passage of fluid and is permeable to the passage of the first drug;

(e) an immediate release coating around the exterior of the microporous membrane;

(f) a first composition in the compartment, said first composition being drug-free for producing a drug-free interval prior to the administration of drug from the compartment;

(g) a second composition in the compartment containing a dose amount of a second drug for producing a therapeutic effect;

(h) a third composition in the compartment that expands in the presence of fluid that enters the device; and

(i) exit means in the semipermeable membrane, external drug layer, and microporous membrane for connecting the exterior of the dosage form with the compartment.

In another aspect, the invention is directed to a method for administering doses of drugs once or twice a day from a single dosage form to the gastrointestinal tract of a warm-blooded animal, which method includes:

(a) admitting the dosage form into the gastrointestinal tract of the warm-blooded animal, said dosage form including:

(1) a compartment;

(2) a semipermeable membrane that surrounds and forms the compartment, said semipermeable membrane comprising a composition that is permeable to the passage of fluid and is substantially impervious to the passage of a drug;

(3) an exterior drug layer containing a dose amount of a first drug on the exterior surface of the semipermeable membrane;

(4) microporous membrane coated around the exterior drug layer said microporous membrane having a composition that is permeable to the passage of fluid and is permeable to the passage of the first drug;

(5) an immediate release coating around the exterior of the microporous membrane

(6) a first composition in the compartment, said first composition being drug-free for producing a drug-free interval prior to the administration of drug from the compartment;

(7) a second composition in the compartment containing a dose amount of a second drug for producing a therapeutic effect;

(8) a third composition in the compartment that expands in the presence of fluid that enters the device; and

(9) exit means in the semipermeable membrane, external drug layer, and microporous membrane for connecting the exterior of the dosage form with the compartment;

(b) releasing the immediate release drug coating by contacting the dosage form with the gastrointestinal fluid;

(c) releasing the exterior drug layer after a first drug free period from initial contact of the dosage form with the gastrointestinal fluid;

(d) imbibing gastrointestinal fluid into the compartment thereby causing the third composition to expand and push against the second compartment; and

(e) releasing the second composition containing the third drug from the compartment after the first composition is released from the compartment.

In preferred embodiments, the semipermeable wall and/or microporous membrane contains a pore-former and the semipermeable wall and/or the microporous membrane contains cellulose acetate. The first and second drugs can be the same or different.

### BRIEF DESCRIPTION OF THE DRAWINGS

FIG. 1 is a view of a dosage system provided by the invention, which dosage form is designed, sized and adapted for admitting into an animal. The dosage system provides time-varying patterns of drug delivery including drug-free release intervals between drug doses;

FIG. 2 is an opened view of FIG. 1 for illustrating the internal structure of the dosage system;

FIG. 3 is an opened view of FIG. 1 depicting a dosage system that provides time-varying patterns of drug delivery including a drug-free interval before release of an exterior drug coating and a second drug-free interval before prolonged drug delivery from the core through a plurality of exit exits;

FIG. 4 is a graph that depicts the hydration coefficient (W_{H}/W_{P}) and osmotic pressure developed by a group of osmotic polymers;

FIG. 5 is a graph of release rate (mg/hr) vs. time (hr) that depicts the diffusion of various pore-formers in cellulose acetate membranes; and

FIG. 6 depicts the release rate pattern for a two-pulse type delivery system.

FIG. 7 depicts an alternative embodiment of a dosage system utilizing a capsule shaped tablet.

In the drawing figures and in the specification, like parts in related figures are identified by like numbers.

### DETAILED DESCRIPTION OF THE INVENTION

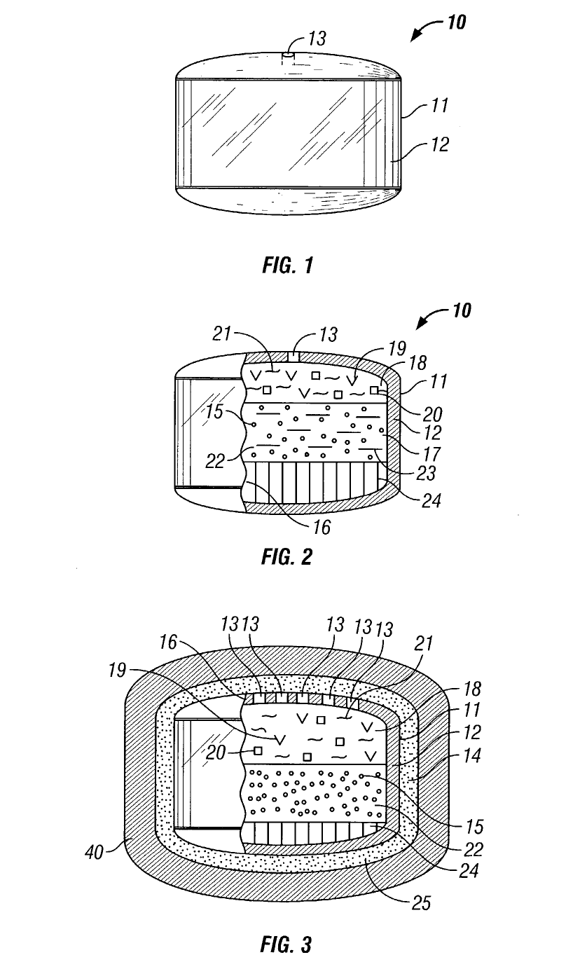

FIG. 1 shows one example of dosage form 10. Dosage form 10 includes a body 11 having a wall 12 that surrounds and forms an internal compartment (not shown). Dosage form 10 further contains at least one exit means 13, for connecting the internal compartment of dosage form 10 with the exterior of dosage form 10.

Dosage form 10 can be manufactured for orally administering a drug 15 to an animal. In another embodiment as shown in FIG. 3, dosage form 10 may contain exterior drug 25. Optionally, dosage form 10 can be sized and shaped for administering drug 25 by the sublingual and buccal routes. The sublingual and buccal routes are typically employed for quicker therapy and can be used when a smaller dose of drug is needed for therapy. The buccal and sublingual routes can also be used to by-pass first-pass hepatic metabolism for direct absorption into the blood stream of drug 25. The sublingual or buccal routes can also be used for administering the first release of drug 25, followed by permitting dosage form 10 to enter the stomach for subsequent drug delivery of drug 15. Drug 15 and drug 25 can be the same or different therapeutic agents.

FIG. 2 shows dosage form 10 manufactured as an osmotic dosage form in opened view at section 16 revealing internal compartment 17. Wall 12 contains at least one exit 13 to connect internal compartment 17 with the exterior of dosage form 10. Within internal compartment 17 are three separate layers: Layer 18, Layer 22, and Layer 24.

Layer 18 is positioned in internal compartment 17 proximal to exit 13 and is drug-free to provide a drug-free interval before drug 15 is released from internal compartment 17. Layer 18 contains a member selected from the group consisting of an osmagent 19, represented by V, and an osmopolymer 20, represented by squares. Layer 18 may optionally contain binder 21 represented by wavy lines. Layer 18 can be manufactured with increased thickness for increasing the drug-free interval of dosage form 10.

Layer 18 may further contain an ionic species such as sodium chloride that functions to salt out with soluble drugs to prevent premature release of such drugs from layer 15.

Layer 22 is positioned in internal compartment 17 between layer 18 and layer 24. Layer 22 contains drug 15, represented by dots. Drug 15 in layer 22 is present with layer former 23, represented by dashes in FIG. 2.

Layer 22 may also contain up to 99 wt % of a polymeric carrier. Layer 22 may also contain a lubricant such as magnesium stearate, corn starch, potato starch, bentonite, citrus pulp, calcium stearate, stearic acid, and the like.

Layer 22 may also contain from about 0 wt % to about 15 wt % of a polyethylene glycol as a solubilizing agent and as a lubricant.

Layer 22 may also contain an osmagent, such as magnesium sulfate, magnesium chloride, potassium sulfate, sodium sulfate, sodium chloride, potassium chloride, and the like. The osmagent in layer 22 imbibes fluid into the layer for enhancing its dispensing from dosage form 10.

The total amount of the components employed in second layer 22 is equal to 100 wt %.

Layer 24 is positioned distal to exit 13 and layer 18, adjacent to layer 22 and is a swellable push layer that contains an osmopolymer that exhibits fluid imbibition properties.

Layer 24 may also contain from about 0 wt % to about 5 wt % of a lubricant such as magnesium stearate, calcium stearate, potassium stearate, lithium stearate, stearic acid and the like; from about 0 wt % to about 3 wt % of a colorant such as red ferric oxide; from about 0 wt % to about 40 wt % of an osmotically effective compound such as magnesium sulfate, magnesium chloride, potassium sulfate, sodium sulfate, lithium sulfate, potassium acid phosphate, mannitol, urea, magnesium succinate, tartaric acid, carbohydrates such as raffinose, sucrose, glucose, sodium chloride, and the like; and from about 0 wt % to about 30 wt % of a binder such as hydroxypropylcellulose, polyvinyl pyrrolidone, polyvinyl alcohol, polyethylene glycol, and the like. The composition of all ingredients present in third layer 24 is equal to 100 wt %.

FIG. 3 shows that dosage form 10 can also have, external drug coat 14 formed on the exterior surface of wall 12. Drug coat 14 is a composition containing from about 1 mg to about 200 mg of drug 25, represented by dots.

FIG. 3 also shows the alternative embodiment of dosage form 10 having outer microporous membrane 40 that comprises totally, or in at least a part, a composition that is permeable to the passage of an exterior fluid present in the environment of use, e.g., gastric fluid, and is substantially permeable to the passage of drug 25 from drug coat 14. Membrane 40 is substantially inert, that is, it maintains its physical and chemical integrity during the dispensing of drug from dosage form 10. Membrane 40 can be formed totally or partially of a member selected from the group consisting of a cellulose ether, cellulose ester, cellulose ester-ether.

Membrane 40 may also optionally contain former 23 as used for wall 12. A preferred method of fabricating membrane 40 uses a pore-former to control the rate of drug flow from drug coat 14 through membrane 40.

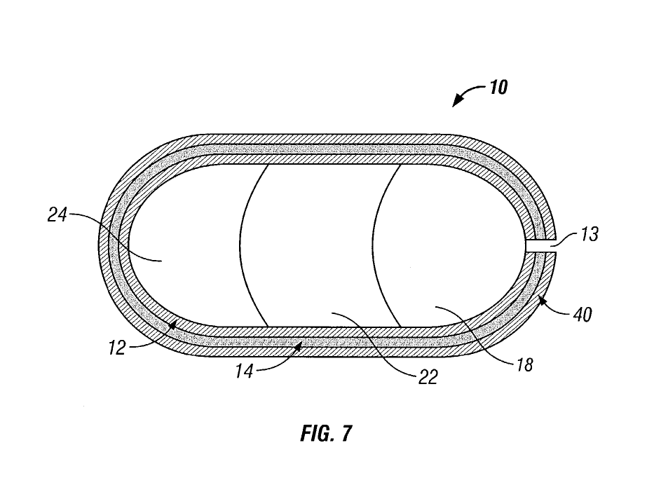

FIG. 7 shows an alternative geometric shape of dosage form 10 that can be utilized, commonly referred to as a capsule-shaped tablet.

The preferred embodiment of the present invention delivers two releases of drug after an initial drug-free interval. The first release is drug 25 delivered from drug coat 14 and the second release is delivered from drug layer 22. Drug 15 is hydrodynamically dispensed through exit 13. The coating formulations are preferably selected such that the resistance to fluid flow through membrane 40 and through drug coat 14 is small relative to the resistance to fluid flow through wall 12. Therefore, the timing of the onset of the second release is due mainly to the permeability and thickness of wall 12 and the mass or thickness of layer 18. Each controlled release may be an extended release or a pulse delivery.

An optional immediate release coating may be added to dosage form 10 to accommodate delivery of third dose of drug. This would then be followed by the drug-free release period.

FIG. 6 illustrates an exemplary drug release pattern. The initial drug-free interval may be shortened or extended before delivery of drug 25 commences. During the initial drug-free interval, exterior fluid from the environment of use imbibes into the drug coat 14 through membrane 40 and dissolves drug coat 14. A portion of the fluid also dissolves the pore-formers within membrane 40. Dissolved drug 25 is then transported evenly through the microporous membrane by diffusion and osmosis, thereby releasing drug 25 over a desired time period, e.g., 1-3 hours.

External fluid is also diverted through wall 12 to hydrate layer 18, layer 22, and layer 24. As this imbibition process continues, layer 24 expands and the drug-free layer 18 is released first followed by the release of drug layer 22. Typically, the initial drug-free interval will be about 0 to 2 hours in length and preferably from about 0.5 hour to about 1 hour in length. Typically, the second drug-free interval, between the two releases, will be about 1 hour to about 10 hours in length and preferably from about 2 hours to 4 hours in length.

Wall 12 of dosage form 10 may partially or completely contain a composition that is permeable to the passage of an external fluid, such as water, present in, for example, the gastro intestinal tract. Wall 12 is substantially impermeable to the passage of drug 15 and other optional ingredients that may be present in internal compartment 17. Wall 12 is substantially inert, in that, it maintains its physical and chemical integrity during the dispensing of drug 15 from dosage form 10.

Wall 12 may be formed completely, or partially of a cellulosic polymer such as cellulose ether, cellulose ester, or cellulose ester-ether. The cellulosic polymers have a degree of substitution (DS) on the anhydroglucose unit, from greater than 0 up to and including 3.

By “degree of substitution,” is meant the average number of hydroxyl groups originally present on the anhydroglucose unit containing the cellulose polymer that are replaced by a substituting group.

Wall 12 of osmotic dosage form 10 can be formed in one technique using the air suspension procedure. This procedure consists of suspending and tumbling the compressed laminate in a current of air and wall forming composition until a wall is applied to the compartment. The air suspension procedure is well-suited for independently forming the wall. The air suspension procedure is described in U.S. Pat. No. 2,799,241; J. Am. Pharm. Assoc., Vol. 48, pp 451 to 459, (1959); and ibid, Vol. 49, pp 82 to 84, (1960).

Osmotic dosage forms can also be coated with a wall-forming composition in a WURSTER® air suspension coater, using methylene dichloride-methanol cosolvent, 80:20, wt:wt, or acetone water cosolvent, 85:15, 90:10, 95:5 wt:wt, or 100:0 using 2.5 to 6% solids. The AEROMATIC® air suspension coater using a methylene dichloride-methanol cosolvent, 87:13, wt:wt, also can be used for applying the wall. Other wall forming techniques such as pan coating system, wall-forming compositions are deposited by successive spraying of the composition on the trilaminate compartment, accompanied by tumbling in a rotating pan. A pan coater is used to produce thicker walls. A more dilute coating solution such as 1.5-2 wt % solids can be used to produce a thinner wall. Finally, the wall coated compartments are dried in a forced air oven at 30° C. to 50° C. for up to a week, or a humidity controlled oven at 50% relative humidity and 50° C. up to 2 to 5 days, to free the dosage form of residual solvent. Generally, the walls formed by these techniques have a thickness of 2 to 20 mils with a presently preferred thickness of 4 to 10 mils.

Representative materials for wall 12 include cellulose acylate, cellulose diacylate, cellulose triacylate, cellulose acetate, cellulose diacetate, cellulose triacetate, mono, di and tricellulose alkanylates, mono, di and tricellulose aroylates, and the like. Exemplary polymers include cellulose acetate having a DS up to and including 1 and an acetyl content up to about 21 weight percent (wt %); cellulose acetate having an acetyl content of about 32 wt % to about 39 wt %; cellulose acetate having a DS of 1 up to and including 2 and an acetyl content of about 21 wt % to about 35 wt %; cellulose acetate having a DS of 2 up to and including 3 and an acetyl content of about 35 wt % to about 45 wt %, and the like. More specific cellulosic polymers include cellulose propionate having a DS of about 1.8 and a propyl content of about 39 wt % to about 45 wt % and a hydroxyl content of about 2.8 wt % to about 5.4 wt %; cellulose acetate butyrate having a DS of 1.8, an acetyl content of about 13 wt % to about 15 wt % and a butyryl content of about 34 wt % to about 39 wt %; cellulose acetate butyrate having an acetyl content of about 26 wt % to about 29 wt %, a butyryl content of about 17 wt % to about 53 wt % and a hydroxyl content of about 0.5 wt % to about 5 wt %; cellulose triacylates having a DS of 2.9 up to and including 3, such as cellulose triacetate, cellulose trivalerate, cellulose trilaurate, cellulose tripalmitate, cellulose trisuccinate, and cellulose trioctanoate; cellulose diacylates having a DS of 2.2 up to and including 2.6, such as cellulose disuccinate, cellulose dipalmitate, cellulose dioctanoate, cellulose dipentanoate, co-esters of cellulose, such as cellulose acetate butyrate and cellulose acetate propionate, and the like.

Additional polymers for wall 12 include acetaldehyde dimethyl cellulose acetate, cellulose acetate ethyl carbamate, cellulose acetate methyl carbamate, cellulose acetate dimethyl aminoacetate, semipermeable polyamides; semipermeable polyurethanes; semipermeable sulfonated polystyrenes; semipermeable cross-linked selectively permeable polymers formed by the coprecipitation of a polyanion and a polycation as disclosed in U.S. Pat. Nos. 3,173,876; 3,276,586; 3,541,005; 3,541,006 and 3,546,142; semipermeable polymers as disclosed in U.S. Pat. No. 3,133,132; semipermeable lightly cross-linked polystyrene derivatives; semipermeable cross-linked poly(sodium styrene sulfonate); and semipermeable cross-linked poly-(vinylbenzyltrimethyl ammonium chloride). The polymers are described in U.S. Pat. Nos. 3,845,770; 3,916,899; and 4,160,020; and in Handbook of Common Polymers by Scott, J. R. and Roff, W. J., (CRC Press, Cleveland, Ohio (1971)).

Wall 12 may optionally contain up to about 30 wt % of a cellulose ether selected from the group consisting of a hydroxypropylcellulose and a hydroxypropylmethylcellulose, and from about 0 wt % to about 30 wt % of a polyethylene glycol. The total weight of all components of wall 12 is equal to about 100 wt %. In a preferred embodiment of dosage form 10, wall 12 contains 3 (mils) of cellulose acetate 398 formulated with about 5 wt % to about 10 wt % of pore former LUTROL® F127.

The term “osmagent”, as used herein, includes osmotically effective solutes, osmotically effective compounds, and osmotic agents. Suitable osmotically effective compounds according to this invention include, inorganic and organic compounds that exhibit an osmotic pressure gradient across wall 12 against an external fluid such as water or gastrointestinal fluids. Osmotically effective compounds useful for the present purpose include magnesium sulfate, magnesium chloride, sodium chloride, lithium chloride, potassium sulfate, sodium carbonate, sodium sulfite, lithium sulfate, potassium chloride, calcium bicarbonate, sodium sulfate, calcium sulfate, potassium acid phosphate, calcium lactate, d-mannitol, urea, inositol, magnesium succinate, tartaric acid, carbohydrates such as raffinose, sucrose, glucose, maltodextrins, α-d-lactose monohydrate, and mixtures thereof. The osmotically effective compound in layer 18 can be in any physical form such as particle, crystal, pellet, tablet, strip, ground, pulverized, film, or granule. The osmotic pressure of saturated solutions of various osmotically effective compounds and for mixtures of osmotic compounds at 37° C. in water, is listed in Table 1. The osmotic pressure can be measured by a commercially available osmometer that measures the vapor pressure difference between pure water and the solution to be analyzed. According to standard thermodynamic principles, the vapor pressure difference is converted into osmotic pressure. In Table 1, osmotic pressures of from 20 atm to 500 atm are set forth. However, the invention includes the use of lower osmotic pressures, and higher osmotic pressures than from 20 atm to 500 atm. The osmometer used for the present measurements is Model 302B, Vapor Pressure Osmometer, manufactured by the Hewlett Packard Co., Avondale, Pa.

TABLE 1

| COMPOUND OR MIXTURE | OSMOTIC PRESSURE (atm) |
|---|---|
| Lactose-Fructose | 500 |
| Dextrose-Fructose | 450 |
| Sucrose-Fructose | 430 |
| Mannitol-Fructose | 415 |
| Sodium Chloride | 356 |
| Fructose | 355 |
| Lactose-Sucrose | 250 |
| Potassium Chloride | 245 |
| Lactose-Dextrose | 225 |
| Mannitol-Dextrose | 225 |
| Dextrose-Sucrose | 190 |
| Mannitol-Sucrose | 170 |
| Sucrose | 150 |
| Mannitol-Lactose | 130 |
| Dextrose | 82 |
| Potassium Sulfate | 39 |
| Mannitol | 38 |
| Sodium Phosphate Tribasic-12H₂O | 36 |
| Sodium Phosphate Dibasic - 7H₂O | 31 |
| Sodium Phosphate Dibasic - 12H₂O | 31 |
| Sodium Phosphate Dibasic - Anhydrous | 29 |
| Sodium Phosphate Monobasic- H₂O | 28 |

For the purpose of this invention, the solubility of an osmagent or a drug in a fluid can be determined by various methods known in the art. One such method includes, preparing a saturated solution of an osmagent or of a drug for example, a fluid plus osmagent or drug, and ascertaining by analysis the amount of osmagent or drug present in a definite quantity of the fluid. A simple apparatus for this purpose consists of a suitable test tube fastened upright in a water bath maintained at constant temperature and pressure, for example at 37.5° C. and 1 atm. The fluid and osmagent or drug are placed in the tube and stirred by a motor driven rotating glass spiral. After a period of stirring, sufficient to reach a saturated solution, a definite weight of the fluid is analyzed. If the analysis shows no increase, after continued stirring, in the presence of excess solid osmagent or drug in the fluid, the solution is saturated and the results are taken as the solubility of the osmagent or drug in the fluid. Numerous other methods are available for the determination of the solubility of the osmagent or the drug in a fluid. Typical methods used for the measurement of solubility are chemical analysis, measurement of density, refractive index, electrical conductivity, and the like.

Details of various methods for determining solubilities are described in, for example, United States Public Health Service Bulletin, No. 67 of the Hygienic Laboratory; Encyclopedia of Science and Technology, 12: 542-556, McGraw Hill, Inc. (1971), Encyclopaedic Dictionary of Physics, 6: 545-557, Pergamon Press Inc. 1962.

Osmopolymer 20 used to form layer 18 contains hydrophilic polymers that are noncross-linked or lightly cross-linked, or cross-links formed by ionic, hydrogen, or covalent bonds. Osmopolymer 20 interacts with water and aqueous biological fluids and forms a solution or a suspension with a high osmotic pressure that is osmotically pumped through exit 13. The hydrophilic polymers can be of plant or animal origin, and can be prepared by modifying naturally occurring structures, and synthetic hydrophilic polymers. Hydrophilic polymers include, for example, poly(hydroxyalkyl methacrylate), poly(N-vinyl-2-pyrrolidone), anionic and cationic hydrogels, polyelectrolyte complexes, poly(vinyl alcohol) having a low acetate residual and cross-linked with a cross-linking agent, such as glyoxal, formaldehyde, glutaraldehyde; methyl cellulose cross-linked with dialdehyde, a mixture of cross-linked agar and carboxymethyl cellulose, a water soluble, water-swellable copolymer produced by forming a dispersion of finely divided copolymer of maleic anhydride with styrene, ethylene, propylene, butylene, or isobutylene cross-linked with from about 0.001 moles to about 0.5 moles of a polyunsaturated cross-linking agent per mole of maleic anhydride in the copolymer, water-swellable polymers of N-vinyl lactams, cross-linked polyethylene oxides, and the like.

Other hydrophilic polymers include those exhibiting a cross-linking of about 0.05 wt % to about 60wt %, hydrophilic hydrogels known as CARBOPOL® acidic carboxy polymer, CYANAMER® polyacrylamides, cross-linked water-swellable indene maleic anhydride polymers, GOOD-RITE® polyacrylic, AQUA-KEEPS® acrylate polymer, diester cross-linked polyglucan, and the like. Useful hydrophilic polymers are also described in U.S. Pat. No. 3,865,108, Hartop et al.; U.S. Pat. No. 4,002,173, Manning et al.; U.S. Pat. No. 4,207,893, Michaelset al.; and in Handbook of Common Polymers (Scott and Roff), published by the Chemical Rubber Company, Cleveland, Ohio.

Other hydrophilic polymers useful for forming layer 18 include agarose, alginates, amylopectin, arabinoglactan, carrageen, eucheuma, fucoidan, furcellaran, gelatin, guar gum, gum agar, gum arabic, gum ghatti, gum karaya, gum tragacanth, hypnea, laminarin, locust bean gum, pectin, polyvinyl alcohol, polyvinyl pyrrolidone, propylene glycol aginates, n-vinyl lactam polysaccharides, xanthan gum, polyethylene oxide, sodium carboxy methylcellulose, and the like. The hydrophilic polymers are known in Controlled Release System, Fabrication Technology, 11:46(1988) published by CRC Press, Inc.

The osmotic pressure of a hydrophilic polymer, i.e., an osmopolymer, or of an osmopolymer osmagent composition, can be determined by measuring the increase in volume and weight of the composition. Measurements are made by placing the composition inside a cup containing a semipermeable wall that surrounds a salt layer and an inner fluid impermeable membrane, which has been immersed in water at 37° C. The osmotic pressure of the composition is determined from the weight gain of the cup compared to a similar cup filled with a saturated solution containing an osmagent, such as sodium chloride, containing excess osmagent. As the osmotic pressure of the osmagent solution is experimentally known, from vapor pressure osmomitry, the osmotic pressure of the osmopolymer is calculated therefrom.

The osmotic pressure generated from an osmotically active solution can be ascertained by the simplified form of Van't Hoff's Law (Equation 1)π=RTiC₂/MW₂, wherein TT is the osmotic pressure generated by an osmotic solute, R is the gas constant, T is the temperature (° K.), C₂ is the osmotic solute concentration in solution (mg/ml), MW₂ is the molecular weight of the solute, and (i) is the number of ionizable species or sites per molecule. For small molecules in which the solubility (S) of the compound can be calculated by substituting C═S, as seen from accompanying Equation 2: π=RTiS/MW₂.

For hydrophilic polymers which are usually miscible in water, the osmotic potential preferably is measured by water imbibition, in which the weight gain of the polymer is contained inside the semipermeable cup described above. The osmotic pressure of the osmopolymer at any degree of water hydration is calculated from the known water permeability of the semipermeable cup according to Equation 3: π=(dv/dt)h/AK, wherein (dv/dt) is the water imbibition rate, (h) is the membrane thickness, (A) is the membrane area, and (K) is the water permeability of the membrane.

The hydration coefficient (W_{H}/W_{P}) is the ratio wherein W_{H} is the weight of water imbibed into the osmopolymer and W_{P} is the weight of the dry osmopolymer.

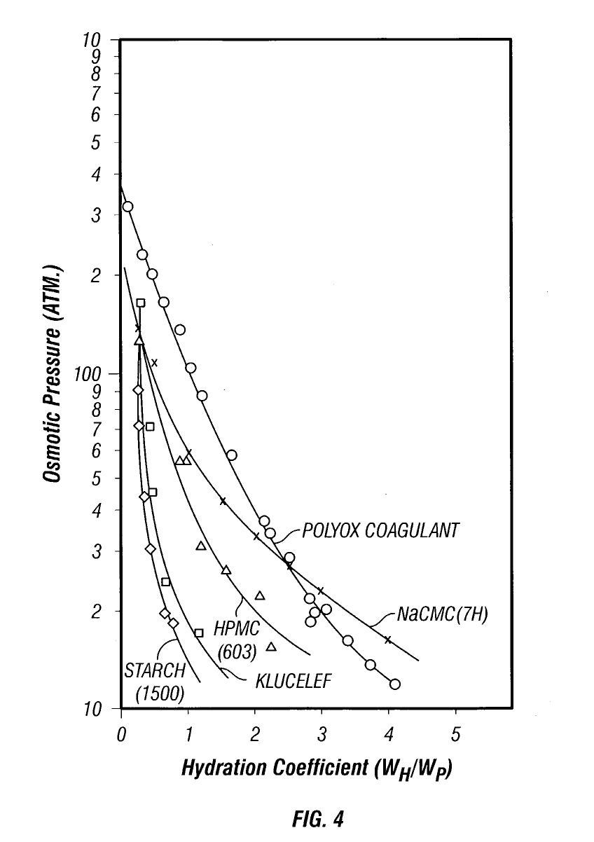

In FIG. 4, KLUCEL EF® denotes hydroxypropycellulose, the letters HPMC denote hydroxypropylmethylcellulose, and POLYOX® COAGULANT denotes polyethylene oxide having a molecular weight of about 5,000,000 daltons, and NaCMC(7H) denotes sodium carboxymethylcellulose.

Layer 18 may exhibit a viscosity from about 100 centipoises to about 10,000,000 centipoises when dosage form 10 is used in vivo, i.e., a temperature of about 35° C. to about 45° C. For example, layer 18 can contain a polyethylene oxide with a molecular weight from about 10,000 daltons to about 7,000,000 daltons; about a 1% solution, such that the viscosity is about 5 centipoises to about 20,000 centipoises at a room temperature of about 23° C. A layer 18 containing polyvinyl pyrrolidone having a molecular weight from about 10,000 daltons to about 5,000,000 daltons, about a 10% solution, has a viscosity that is about 5 centipoises to about 5000 centipoises at 25° C. A layer 18 containing hydroxypropylmethylcellulose having a molecular weight from about 9,000 daltons to about 241,000 daltons, about a 2% solution has a viscosity that is about 3 centipoises to about 100,000 centipoises at 20° C. The viscosity of layer 18, (or the viscosity of other compositions) is ascertained by conventional measurements. The viscosity, or the resistance of a composition to flow when it is subjected to a shear stress can be measured by a Wells-Brookfield Viscometer. Methods for measuring viscosity are known in Pharmaceutical Sciences, by Remington, 14th Ed., pp. 359-71, (1970), published by Mack Publishing Co., Easton, Pa.

The term “drug” as used herein, includes any physiologically or pharmacologically active substance that produces a local or systemic effect in animals, such as mammals; avians; fishes and reptiles. The term “physiologically,” as used herein, denotes the administration of a drug to produce normal levels and functions.

The term “pharmacologically”, as used herein, denotes variations in response to the amount of drug administered to an animal. See Stedman's Medical Dictionary, 1966 published by Williams and Wilkins, Baltimore, Md.

Drug 15 and drug 25 can be the same or different drugs. Representative drugs that can be delivered to an animal include, for example, inorganic and organic compounds such as anticonvulsants, analgesics, anti-Parkinsons, anti-inflammatories, calcium antagonists, anesthetics, antimicrobials, antimalarials, antiparasites, antihypertensives, antihistamines, antipyretics, alpha-adrenergic agonist, alpha-blockers, biocides, bactericides, bronchial dilators, beta-adrenergic blocking drugs, contraceptives, darciovascular drugs, calcium channel inhibitors, depressants, diagnostics, diuretics, electrolytes, hypnotics, hormonals, hyperglycemics, muscle contractants, muscle relaxants, ophthalmics, psychic energizers, parasympathomimetics, sedatives, sympathomimetics, tranquilizers, urinary tract drugs, vaginal drugs, vitamins, nonsteroidal anti-inflammatory drugs, angiotensin converting enzymes, polypeptides and the like.

These drugs may act upon one or more of the following systems: the peripheral nerves, adrenergic receptors, cholinergic receptors, nervous system, skeletal muscles, cardiovascular system, smooth muscles, blood circulatory system, synaptic sites, neuroeffector junctional sites, endocrine system, hormone systems, immunological system, reproductive system, skeletal system, autacoid systems, alimentary and excretory systems, inhibitory of autocoid systems, alimentary and excretory systems, inhibitory of autocoids and histamine systems.

These drugs may be soluble in water, greater than about 400 mg/μl. Examples of such soluble drugs include prochlorperazine edisylate, ferrous sulfate, aminocaproic acid, potassium chloride, mecamylamine hydrochloride, procainamide hydrochloride, amphetamine sulfate, benzphetamine hydrochloride, isoproterenol sulfate, methamphetamine hydrochloride, phenmetrazine hydrochloride, bethanechol chloride, methacholine chloride, pilocarpine hydrochloride, atropine sulfate, apoatropine HCl, scopolamine bromide, isopropamide iodide, trihexethyl chloride, phenformin hydrochloride, methylphenidate hydrochloride, cimetidine hydrochloride, theophylline cholinate, cephalexin hydrochloride, venlafaxine HCl, metformin HCl, and the like.

These drugs may also be insoluble in water, less than about 400 mg/μl. Examples of such insoluble drugs include, for example, diphenidol, meclizine hydrochloride, prochlorperazine maleate, phenoxybenzamine, thiethylperazine maleate, anisindione, diphenadione erythrityl tetranitrate, digoxin, isofulrophate, acetazolamide, methazolamide, bendroflumethiazide, chlorpropamide, tolazamide, chlormadinone acetate, phenaglycodol, allopurinal, aluminum aspirin, methotrexate, acetyl sulfisoxazole, erhtyromycin, progestins, estrogenic, progestational, corticosteroids, hydrocortisone, hydrocorticosterone acetate, cortisone acetate, triamcinolone, methylesterone, 17 beta-estradiol, ethinyl estradiol, ethinyl estradiol 3-methyl ether, prednisolone, 17 beta-hydroxyprogesterone acetate, 19-nor-progesterone, norgestrel, norethindrone, norethisterone, norethiderone, progesterone, norgesterone, norethynodrel, phenytoin, and the like.

Examples of other drugs that can be delivered by dosage form 10 include, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chloropromaxine, methyldopa, dihydroxyphenylalanine, pivaloxyethyl ester of alpha-methyldopa hydrochloride, theophylline, calcium gluconate, ketoprofen, ibuprofen, cephalexin, erythromycin, haloperidol, zomepirac, ferrous lactate, vincamine, verapamil, midazolam, diazepam, phenoxybenzamine, diltiazem, milrinone, mandol, guanabenz, hydrochlorothiazide, ranitidine, flurbiprofen, fenbufen, fluprofen, tolmetin, alclofenac, mefenamic, flufenamic, diflunisal, minodipine, nitredipine, nisoldipine, nicardipine, felodipine, lidoflazine, tiapamil, gallopamil, amlodipine, mioflazine, lisinopril, analapril, captopril, ramipril, endlapriat, famotidine, nizatidine, sucralfate, etintidine, tertatolol, minoxidil, chlordiazepoxide, chlordiazepoxide, amintriptylin hydrochloride, imipramine hydrochloride, imipramine pamoate, ranitidine HCl, tolteradine HCl, and the like.

Still other drugs that can be delivered by dosage form 10 may include drugs that are administered in the colon to produce a therapeutic effect. Such drugs include, for example, drugs conventionally used in the treatment of colitis, ulcerative colitis, Crohn's disease, idiopathic prototis and other diseases of the colon. Representative drugs include, for example, salicylazosulfapyridine, also known as sulfasalazine and salazopyrin; adrenocorticosteroids, such as hydrocortisone, prednisolone phosphate, prednisolone sulfate, prednisone, prednisolone metasulphobenzoate sodium, prednisolone sodium phosphate and the like; corticosteroids such as beclomethasone, beclomethasone acetate, beclomethasone valerate, beclomethasone propionate, beclomethasone diproprionate, and the like; and cyclosporin. In another aspect, drug 15 also includes drugs for the treatment of irritable bowel syndrome. Additionally, drug 15 can be selected to alter bowel motility and fluid absorption. Such drugs are represented by calcium channel blocking drugs, opiods, anticholinergics and benzodiazepides.

Other drugs include non-steroidal anti-inflammatories, and analgesic drugs. Such drugs include, for example, a nonsteroidal propionic acid derivative, a nonsteroidal acetic acid derivative, a nonsteroidal fenamic acid derivative, a nonsteroidal biphehylcarboxylic acid derivative, and nonsteroidal axicam derivatives. The non-steroidal propionic acid derivatives include, for example, benoxaprofen, carprofen, flurbiprofen, fenoprofen, fenbufen, ibuprofen, indoprofen, ketoprofen, naproxen, miroprofen, oxaprozin, pranoprofen, pirprofen, suprofen, tiaprofenic acid, fluprofen, alminoprofen, bucloxic acid and the like. The non-steroidal acetic acid derivatives include, for example, alclofenac acematacin, aspirin, diclofenac, indomethacin, ibufenac, isoxepac, furofenac, fentiazac, clidanac, oxpinac, sluinda, tolmetin, zomepirac, zidometacin, tenclofenac, tiopinac, and the like. The non-steroidal fenamic acid derivatives include, for example, mefenamic acid, fufenamic acid, niflumic acid, meclofenamic acid, tolfenamic acid, and the like. Representative biphenylcarboxylic carboxylic acid nonsteroid derivatives include, for example, diflunisal, flufenisal, and the like. Representative nonsteroidal oxicam derivatives include, for example, isoxicam, piroxicam, sudoxicam, and the like. Other useful drugs include potassium chloride, potassium carbonate, and the like.

Drug 15 in layer 22 can be present in various forms, such as uncharged molecules, molecular complexes, pharmacologically acceptable salts such as hydrochloride, hydrobromide, sulfate, laurate, palmitate, phosphate, nitrite, borate, acetate, maleate, tartrate, oleate and salicylate. For acidic drugs, salts of metals, amines or organic cations; for example, quaternary ammonium can be used. Derivatives of drugs such as ester, ethers and amides can also be used. Additionally, a drug that is water insoluble can be used in a form that is a water soluble derivative thereof to serve as a solute, and on its release from the dosage form, is converted by enzymes, hydrolyzed by physiological pH or other metabolic processes to the original biologically active form. The amount of drug in dosage form 10 is generally from about 0.05 ng to about 50 g or more, with individual devices containing, for example, from about 20 ng to about 2.0 g, i.e, 25 ng, 1 mg, 5 mg, 10 mg, 25 mg, 125 mg, 500 mg, 750 mg, 1.0 g, 1.2 g, 1.5 g, and the like. The dosage form 10 can be administered for example, once, twice or thrice daily to an animal.

Former 23 contains, for example, a polymeric carrier of a water soluble gum such as carrageenan, fucoidan, gum ghatti, tragacanthin, arabinoglactin, pectin, xanthan, and the like; water soluble salts of polysaccharides such as maltodextrins, sodium alginate, sodium tragacanthin, hydroxyalkylcellulose wherein the alkyl member is either a straight or branched chain of 1 to about 7 carbon atoms such as hydroxymethylcellulose, hydroxyethylcellulose, hydroxypropylcellulose, and the like; synthetic water-soluble cellulose-based layer formers, such as methyl cellulose and its hydroxyalkyl methylcellulose derivatives, such as hydroxyethyl methylcellulose, hydroxypropyl methylcellulose, hydroxybutyl methylcellulose, polyoxyethylene having a molecular weight of about 50,000 daltons to about 8,000,000 daltons and present as a polyoxyethylene concentration from about 0 wt % to about 25 wt % and the like; and other cellulose polymers such as carboxymethylcellulose.

Former 23 may contain polyvinyl pyrrolidone (a blend of gelatin and polyvinyl pyrrolidone), glucose, zinc palmitate, aluminum stearate, amgnesium oleate, pulverized teflon, poly(lactide), poly(glycolide), poly(lactide-glycolide) copolymers, halogenated vegetable oil, pulverized talc, and the like.

Osmopolymers are hydrophilic polymers that are swellable and exhibit fluid imbibition properties sufficient to form an expandable push layer 24 is dosage from 10. Hydrophilic polymers used as osmopolymers interact with water and aqueous biological fluids and swell or expand to an equilibrium state and retain a significant portion of the imbibed water within the polymer structure. The hydrophilic polymers swell or expand to a high degree, typically exhibiting a 2 to 60 fold volume increase. The hydrophilic polymers can be noncross-linked or cross-linked. The swellable, hydrophilic polymers are, in one embodiment, lightly cross-linked. Typically, such cross-links are formed by covalent bonds or residue crystalline regions after swelling. The hydrophilic polymers can be synthetic, or derived from a plant or animal. Suitable hydrophilic polymers include, for example, poly(hydroxy-alkyl methacrylate) having a molecular weight from about 30,000 daltons about 5,000,000 daltons; poly(vinyl-pyrrolidone) having a molecular weight from about 10,000 daltons to about 360,000 daltons; anionic and cationic hydrogels; polyelectrolyte complexes; poly(vinyl alcohol) having a low acetate residual, cross-linked with glyoxal, formaldehyde, or glutaraldehyde having a degree of polymerization from about 200 monomer units to about 30,000 monomer units; a mixture of methyl cellulose, cross-linked agar and carboxymethyl cellulose; a mixture of hydroxypropyl methylcellulose and sodium carboyxmethylcellulose, hydroxypropyl methylcellulose; a water insoluble, water swellable copolymer reduced by forming a dispersion of finely divided copolymer of maleic anhydride with styrene, ethylene, propylene, butylene or isobutylene cross-linked with from about 0.001 moles to about 0.5 moles of saturated cross-linking agent per mole of maleic anhydride in copolymer; water swellable polymers of N-vinyl lactams; polyoxyethylene-polyoxypropylene gel; polyoxybutylene-polyethylene block copolymer gel; carbo gum, polyacrylic gel; polyester gel; polyuria gel; polyether gel; polyamide gel; polyamide gel; polypeptide gel; polyamine acid gel; polycellulosic gel; polygum gel; initially drug hydrogels that generally imbibe and absorb water which penetrates the glassy hydrogel and lowers its glass transition temperature; and the like.

Other hydrophilic polymers include polymers that form hydrogels, such as CARBOPOL® acidic carboxy polymers, polyacylic acid cross-linked with polymer having a molecular weight of about 250,000 daltons to about 4,000,000 daltons CYANAMER® polyacrylamides; cross-linked water swellable indene-maleic anhydride polymers; GOOD-RITE® polyacrylic acid having a molecular weight of about 80,000 daltons to about 200,000 daltons; POLYOX® polyethylene oxide polymers having a molecular weight of about 100,000 daltons to about 8,500,000 daltons; starch graft copolymers; AQUA-KEEPS® acrylate polymer polysaccharides composed of condensed glucose units such as diester cross-linked polyglucan; and the like. Representative polymers that form hydrogels are known and are described in, for example, U.S. Pat. Nos. 3,865,108, 4,002,173, 4,207,893, and the Handbook of Common Polymers, by Scott and Roff, published by the Chemical Rubber Company, Cleveland, Ohio.

The amount of osmopolymer in push layer 24 is typically from about 0.01% to about 99%. In one embodiment, the osmopolymer composition in layer 24 exhibits a lesser osmotic pressure than the osmotic pressure in layer 22 and layer 18. As a result, during operation of dosage form 10, fluid imbibed into layer 24 creates a greater viscosity than that created in layer 22. Similarly layer 22 exhibits a greater viscosity than layer 18. The present invention provides a sequential viscosity gradient (N) according to Equation 4, wherein (1) denotes layer 18, (2) denotes layer 22 and (3) denotes layer 24.

N₍₃₎>N₍₂₎>N₍₁₎

The term “exit” includes aperture, orifice, bore, pore, porous element through which the drug can be pumped, diffuse, travel or migrate, hollow fiber, capillary tube, porous overlay, porous insert, microporous member, and the like.

The term exit also includes a material that erodes or is leached from wall 12 in the fluid environment of use to produce at least one exit in dosage form 10. Representative materials include an erodible poly(glycolic) acid or poly(lactic) acid member in the wall; a gelatinous filament; poly(vinyl alcohol); leachable materials such as fluid removable pore forming polysaccharides, salts, or oxides, and the like.

An exit or a plurality of exits can be formed by leaching a material such as sorbitol, sucrose, lactose, fructose or the like, from the wall. The exit can have any shape such as round, triangular, square, elliptical, and the like, for assisting in the metered released of drug from dosage form 10. Dosage form 10 can be constructed with one or more exits in spaced apart relation on one or more than a single surface of a dosage form.

Exits and equipment for forming passages are disclosed in U.S. Pat. Nos. 3,845,770 and 3,916,899; in U.S. Pat. No. 4,063,064; and in U.S. Pat. No. 4,088,866. Osmotic exits of controlled drug releasing dimension, sized, shaped and adapted as a drug releasing pore formed by leaching to provide a drug-releasing pore of controlled osmotic release rate are disclosed in U.S. Pat. No. 4,200,098; and in U.S. Pat. No. 4,285,987.

Exit 13 is preferably fabricated by a continuous aperture passing through wall 12, coat 14, and outer microporous membrane 40, connecting layer 18 of internal compartment 17 to the exterior of dosage form 10.

Drug 25 is preferably blended with an aqueous soluble film-forming carrier such as methylcellulose, hydroxypropylcellulose, hydroxypropylmethylcellulose, optionally blended with a plasticizer such as polyethylene glycol (PEG) or acetylated triglycerides or the like. A preferred drug coat 14 is a film of 1-3 mils of hydroxyethylcellulose or hydroxypropyl methylcellulose containing from about 0.1 wt % to about 40 wt % of drug 25. When employing hydroxypropylmethyl cellulose preferably 5-30% PEG is used to promote flexibility and as an anti-tack agent in film coating operators. Coat 14 releases drug 25 according to the programmable delivery patterns provided by dosage form 10.

Membrane 40 can be fabricated using materials generally described as having a sponge-like appearance that provides a supporting structure for interconnected pores or voids. The material can be isotropic wherein the structure is homogeneous throughout a cross-sectional area, the material can be anisotropic wherein the structure is non-homogeneous throughout a cross-sectional area, or the materials can have both cross-sectional areas. The materials are opened-celled, as the pores are substantially continuous or connected pores having an opening on both faces of membrane 40. The pores maybe interconnected through paths of regular and irregular shapes including curved, linear, curved-linear, randomly oriented continuous pores, hindered connected pores and other interconnected porous paths discernible by microscopic examination. Techniques for preparing microporous membranes are further described in U.S. Pat. No. 4,692,336 which is incorporated herein by reference.

Membrane 40 is characterized as having a reduced bulk density as compared to the bulk density of the corresponding non-porous precursor microporous membrane. The morphological structure of the total microporous membrane will have a greater proportion of total surface area than the non-porous membrane. The microporous membrane can be further characterized by the pore size, the number of pores, the tortuosity of the microporous paths, and the porosity which relates to the size and the number of pores. The pore size of a microporous membrane is easily ascertained by measuring the observed pore diameter at the surface of the material under the electron microscope. Generally materials possessing from about 5% to about 95% pores, and having a pores size of from about 10 angstroms to about 100 microns can be used for making membrane 40.

Microporous membrane materials are commercially available and they can be made by methods well known in the art. The materials can be made by etched nuclear tracking; by cooling a solution of a flowable polymer below the freezing point whereby the solvent evaporates from the solution in the form of crystals dispersed in the polymer, and then curing the polymer followed by removing the solvent crystals; by cold stretching or hot stretching at low or high temperatures until pores are formed; by leaching from a polymer a soluble component by an appropriate solvent; by ion exchange reaction; and by polyelectrolyte processes.

In a presently preferred embodiment, the microporous membrane is formed in the environment of use from a precursor microporous membrane. This latter membrane contains a pore-former that is removed from the precursor by dissolving or leaching a pore-former therefrom, thus forming an operable microporous membrane. The pore-formers useful for the present purpose are a member selected from the group consisting of about 1 to 50%, or more by weight of a solid pore-former, about 0 to 20% percent by weight of a liquid pore-former, and mixtures thereof. In another embodiment, the microporous membrane can be formed by a compression coating technique. In this latter embodiment, a rigid microporous wall substantially free of substances soluble or swellable in the fluid present in the environment of use can be formed by compression coating a microporous material around the compartment forming ingredients. Generally a microporous membrane is formed under a compression pressure of 500 to 5000 kg/cm², usually in a rotary machine.

Materials suitable for forming microporous membrane 40 include polycarbonates comprising linear polyesters of carbonic acid in which carbonate groups recur in polymer chains by phosgenation of a dihydroxy aromatic such as a bisphenol, microporous poly(vinyl chloride), microporous polyamides such as polyhexamethylene adipamide, microporous modacrylic copolymers including those formed of polyvinylchloride and acrylonitrite, styrene-acrylic acid copolymers, microporous polysulfones characterized by diphenylene sulfone groups in the linear chain thereof, halogenated polymers such as polyvinylidene fluoride, polyvinylfluoride and polyfluorohalocarbon, polychloroethers, cellulose esters, cellulose ethers, cellulose acylates, acetal polymers such as polyformaldehyde, polyesters prepared by esterification of a dicarboxylic acid or anhydride with a polyol, poly(alkylenesulfides), phenolic polyesters, microporous poly(saccharides) having substituted and unsubstituted anhydroglucose units, asymmetric porous polymers, cross-linked olefin polymers, hydrophobic and hydrophilic microporous homopolymers, copolymers or interpolymers having a reduced bulk density.

Additional microporous membrane materials include materials that are substantially insoluble in the fluid present in the environment of use, are inert, non-disintegrating, noneroding and are materials that can be compressed in powder form, applied by air suspension, dipping techniques, and the like. Exemplary materials include ethyl cellulose, poly(urethanes), copolymers of divinyl chloride and acrylonitrile, organic materials such as crosslinked chain-extended poly(urethanes), microporous poly(urethanes) in U.S. Pat. No. 3,524,753; poly(imides), poly(benzimidazoles), collodion (cellulose nitrate with 11% nitrogen), regenerated proteins, microporous materials prepared by diffusion of a multivalent cations into polyelectrolyte sols in U.S. Pat. No. 3,565,259, anisotropic microporous materials of ionically associated polyelectrolytes, microporous polymers formed by the coprecipitation of a polycation and a polyanion as described in U.S. Pat. Nos. 3,276,589; 3,541,006; and 3,546,142, derivatives of poly(styrene) such as poly(sodium styrene sulfone) and poly(vinylbenzyltrimethyl-ammonium chloride), the microporous materials discussed in U.S. Pat. Nos. 3,615,024; 3,646,178; and 3,852,224, the microporous walls having a plurality of micropores as disclosed in U.S. Pat. No. 3,948,254; and the like.

The term “pore-former,” includes pore-forming solids and pore-forming liquids. In the later expression, the term, “liquid” generically embraces semi-solids, pastes and viscous fluids. The pore-formers can be inorganic or organic. The term, “pore-former” for both solids and liquids include substances that can be dissolved, extracted or leached from the precursor microporous wall by fluid present in the environment of use to form an operable, open-celled type microporous membrane.

Preferred liquid pore-formers are liquid at room temperature and they include, for example, polyethylene glycol with a molecular weight of less than 600 grams per mole, glycerin, triacetin, citric acid esters such as triethyl citrate, acetyl triethyl citrate, tributyl citrate and acetyl tributyl citrate, dibutyl sebacate, and the like.

Additionally, the pore-formers suitable for the invention include pore-formers that can be dissolved, leached, or extracted without causing physical or chemical changes in the polymer. The pore-forming solids can have a size of about 0.1 to 200 microns and include alkali metals salts such as lithium chloride, lithium carbonate, sodium chloride, sodium bromide, sodium carbonate, potassium chloride, potassium sulfate, potassium phosphate, sodium benzoate, sodium acetate, sodium citrate, potassium nitrate; the alkaline earth metal salts such as calcium phosphate, calcium nitrate, calcium chloride; the transition metal salts such as ferric chloride, ferrous sulfate, zinc sulfate, cupric chloride, manganese fluoride, manganese fluorosilicate; organic compounds such as polysaccharides including sucrose, glucose, fructose, mannitol, mannose, galactose, aldohexose, altrose, talose, sorbitol; organic aliphatic and aromatic ols including diols, polyols; organic ols including diols and polyols, and other polyols such as polyhydric alcohol, polyalkylene glycol, polyglycol, poly (α-ω)alkylenediols, and the like.

The pore-formers are nontoxic and on their removal from the wall, channels are formed through the wall, that fills with fluid. The channels become, in one embodiment, means or paths for releasing drug 25. The pores extend from the inside wall to the outside wall for effective release of drug 25 to the exterior of the delivery system. In a presently preferred embodiment, membrane 40 contains 1 to 50 wt % of pore-former selected from the group consisting of inorganic salts, organic salts, carbohydrates, and ols that are used when the pores of controlled porosity are formed during use in a biological environment.

Preferred pore-formers include the nonionic series of difunctional block-polymers terminating in primary hydroxyl groups and which are commercially available under the trademark LUTROL® as manufactured by BASF. These pore-formers can be incorporated into membrane 40 as part of the dosage form. Upon contact with the gastric fluid in a patient, these pore-formers will elute from membrane 40 to leave pores.

Microporous membranes incorporating different LUTROL® pore-formers were tested for the rate at which the pore-formers eluted from the membrane. Five mils thick membranes comprising about 70 wt % cellulose acetate (CA 398-10) and about 30 wt % pore-former selected from either LUTROL® F68, F87, F108, or F127 were placed into vials containing 15 ml water at 37^{□} C.

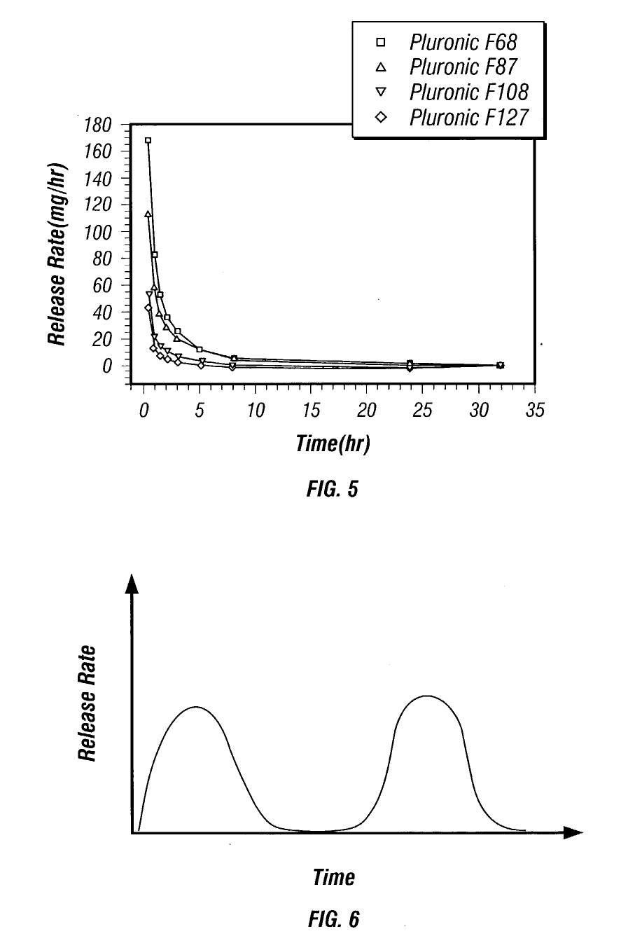

FIG. 5 shows the diffusion rates of the different pore-formers. LUTROL® F68 elutes from cellulose acetate membranes much faster than other types of LUTROL® whereas the F127 grade elutes more slowly. Blends of fast-eluting and slow-eluting pore-formers can add an element of rate control to delay onset of the initial pulse of drug 25.

A preferred microporous membrane comprises a 4 mils thick layer of cellulose acetate 398 containing about 20 wt % LUTROL® F68 and about 25 wt % LUTROL® F127.

Dosage form 10 of the invention is manufactured by standard manufacturing techniques. For example, in one manufacture, the beneficial drug and other ingredients comprising drug layer 22 are blended and pressed into a solid layer. The drug and other ingredients can be blended also with a solvent and mixed into a solid or semisolid formed by conventional methods such as ball-milling, calendering, stirring or rollmilling and then pressed into a preselected shape. Drug layer 22 possesses dimensions that correspond to the internal dimensions of the area the layer is to occupy in the dosage form and it also possesses dimensions corresponding to layer 18 for forming a contacting arrangement therewith.

Next, the osmopolymer, hydrogel or push layer 24, is placed in contact with drug layer 22. The osmopolymer layer is manufactured using techniques for providing the drug layer. Delay layer 18 is manufactured using similar procedures.

The layering of osmopolymer layer 24, delay layer 18, and drug layer 22 can be fabricated by conventional press-layering techniques.

Finally, the three-layer compartment forming members are surrounded and coated with wall 12.

Exit 13 is laser drilled through wall 12 to contact delay layer 18, with the dosage form optically oriented automatically by the laser equipment to form exit 13 on the preselected surface.

In another manufacture, the dosage form is manufactured by the wet granulation technique. In the wet granulation technique, for example, the drug and the ingredients comprising the drug layer are blended using an organic solvent, such as isopropyl alcohol-ethylene dichloride 80:20 v:v (volume:volume) as the granulation fluid. Other granulating fluid such as 100% denatured alcohol can be used for this purpose. The ingredients forming the drug layer are individually passed through a 40 mesh screen and then thoroughly blended in a mixer. Next, other ingredients comprising the drug layer are dissolved in a portion of the granulation fluid, such as the cosolvent described above. Then, the latter prepared wet blend is slowly added to the drug blend with continual mixing in the blender. The granulating fluid is added until a wet blend is produced, which wet mass then is forced through a 20 mesh screen onto oven trays. The blend is dried for 18 to 24 hours at 30° C. to 50° C. The dry granules are sized then with a 20 mesh screen. Next, a lubricant is passed through an 80 mesh screen and added to the dry screen granule blend. The granulation is put into milling jars and mixed on a jar mill for 1 to 15 minutes. The delay layer and the push layers are made by the same wet granulation techniques. The compositions are pressed into their individual layers in a KORSCH® or MANESTY® press-layer press.

Another manufacturing process that can be used for providing the compartment-forming composition layers comprises blending the powdered ingredients for each layer independently in a fluid bed granulator. After the powdered ingredients are dry blended in the granulator, a granulating fluid, for example, poly(vinyl-pyrrolidone) in water, or in denatured alcohol, or in 95:5 ethyl alcohol/water, or in blends of ethanol and water is sprayed onto the powders. Optionally, the ingredients can be dissolved or suspended in the granulating fluid. The coated powders are then dried in a granulator. This process granulates all the ingredients present therein while adding the granulating fluid. After the granules are dried, a lubricant such as stearic acid or magnesium stearate is added to the granulator. The granules for each separate layer are then pressed in the manner described above.

The osmotic device of the invention is manufactured in another embodiment by mixing a drug with composition forming ingredients and pressing the composition into a solid lamina possessing dimensions that correspond to the internal dimensions of the compartment. In another embodiment the drug and other drug composition-forming ingredients and a solvent are mixed into a solid, or a semisolid, by conventional methods such as ballmilling, calendering, stirring or rollmilling, and then pressed into a preselected lamina forming shape. Next, a lamina of a composition comprising an osmopolymer and an optional osmagent are placed in contact with the lamina comprising the drug lamina. Then, a lamina of a composition comprising a drug-free lamina is placed in contact with the other side of the drug lamina and the three lamina comprising the trilaminate surrounded with a semipermeable wall.

The lamination of the middle drug lamina, the first delay lamina and the third push lamina comprising the osmopolymer and optional osmagent composition can be accomplished by using a conventional layer tablet press technique.

The wall can be applied by molding, spraying or dipping the pressed shapes into wall forming materials.

Another and presently preferred technique that can be used for applying the wall is the air suspension coating procedure. The procedure suspends and tumbles the two layered laminate in a current of air until the wall forming composition surrounds the laminate. The air suspension procedure is described in U.S. Pat. No. 2,799,241; J. Am. Pharm. Assoc., Vol, 48, pp 451-459 (1979); and, ibid, Vol. 49, pp 82-84 (1960). Other standard manufacturing procedures are described in Modern Plastics Encyclopedia, Vol. 46, pp 62-70 (1969); and in Pharmaceutical Science, by Remington, 14th Ed., pp 1626-1979, (1970), published by Mack Publishing Co., Easton, Pa.

Exemplary solvents suitable for manufacturing the wall, the laminates and laminae include inert inorganic and organic solvents. The solvents broadly include members selected from the group consisting of aqueous solvents, alcohols, ketones, esters, ethers, aliphatic hydrocarbons, halogenated solvents, cyclaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane ethylene glycol monoethyl ether, ethylene glycol monoethyl acetate, methylene dichloride, ethylene dichloride, propylene dichloride, carbon tetrachloride, chloroform, nitroethane, nitropropane, tetrachoroethan, ethyl ether, isopropyl ether, cyclohexane, cyclo-octane, benzene, toluene, naphtha, tetrahydrofuran, diglyme, aqueous and nonaqueous mixtures thereof, such as acetone and water, acetone and methanol, acetone and ethyl alcohol, methylene dichloride and methanol, and ethylene dichloride and methanol.

### DETAILED DISCLOSURE OF EXAMPLES OF THE INVENTION

The following examples are merely illustrative of the present invention and they should not be considered as limiting the scope of the invention in any way as these examples and other equivalents thereof will become apparent to those versed in the art in the light of the present disclosure and drawings.

### EXAMPLE 1

A dosage form adapted, designed and shaped as an osmotic drug delivery system is manufactured as follows: first 2,500 g of polyethylene oxide having a molecular weight of about 100,000 is mixed with 6,450 g of sorbitol and 1,000 g sodium chloride in a HOBART® mixer at slow speed for 20 minutes. Then, 4 liters of denatured ethanol is slowly added to the above mixer and the mixing continued for an additional 5 minutes to produce a wet granulation. Next, the wet granulation is dried at 31° C. in an oven for 16 hours, and after cooling to room temperature it is passed through a 20 mesh screen. Finally, 50 g of magnesium stearate is added to the granulation and all ingredients are mixed in a twin-shell blender for 1 to 3 minutes, to yield a drug-free granulation composition.

Next, 7,000 g of verapamil HCl, 2,500 g of polyethylene oxide, having a molecular weight of about 200,000 and 450 g of polyvinyl pyrrolidone are mixed in a HOBART® mixer at slow speed for 30 minutes. Then, 3.6 liters of anhydrous ethanol is added slowly to the above mixer and the mixing continued for an additional 4 minutes to yield a wet granulation. Next, the wet granulation is passed through a 7 mesh screen in a FLUID-AIR® mill at 600 rpm, followed by drying the granules at 30° C. in a forced air oven for 18 hours. The dry granules next are passed through a 7 mesh screen in a FLUID-AIR® mill jacketed with chilled water(4° C.) at 550 rpm. Finally, 50 g of magnesium stearate is added to the granulation and al the ingredients mixed in a V-blender for 3 minutes to yield a drug granulation composition.

Next, an osmotic or push layer is prepared by passing separately through a 40 mesh screen the following ingredients: 8,470 g of sodium carboxymethyl cellulose with a molecular weight of 700,000, 940 g of hydroxypropyl cellulose with a molecular weight of 60,000, 470 g of sodium chloride and 100 g of ferric oxide. All of the screened ingredients are then thoroughly mixed in a mixer to yield a homogeneous mix. Then, with continuous mixing, 40 ml of denatured anhydrous ethanol are slowly added and mixing is continued for 2 more minutes to yield a wet granulation.

Next, the wet granulation is passed through a 20 mesh screen, dried at room temperature for 16 hours and passed again through a 20 mesh screen. Finally, 20 g of magnesium stearate is poured through a #60 mesh sieve and then added to the granulation and the ingredients mixed in a roller mill for 3 minutes to yield an osmotic granulation composition.

A three-layered Manesty tablet press is used for forming the three-layer laminate. The press is set with 7/16 inch diameter dies and standard concave punches. First, 200 mg of the drug-free composition is added to the die and tamped, then, 214 mg of the second or drug composition is added to the die and tamped, and then, 120 mg of the third or osmotic composition is added to the die and the three laminae are compressed at 2 ton compression pressure to yield the three laminae in contacting laminated arrangement.

Next, the laminates are surrounded with a semipermeable wall. The wall-forming composition comprises 70% cellulose acetate having an acetyl content of 39.8% and 30% LUTROL® F68 with a molecular weight of 8,400. The wall-forming composition is dissolved in 100% acetone solvent to make a 4% solids solution. The wall-forming composition is sprayed onto and around the laminates in an Hl-COATER® pan coater. Next, two 40 mil exit ports are drilled on the drug-free or delay-layer side of the dosage form. The dosage form is then placed in a 50° C. forced air oven for 3 days to remove residual coating solvent. Finally, the coated laminates are dried for 48 hours in a humidity oven set at 50% relative humidity and 50° C. to remove the coating solvents. The coated wall surrounding the laminate weighed 28 mg.

Next, an exterior drug coating comprising 60 g verapamil HCl and other exterior forming lamina ingredients comprising 40 g hydroxypropyl cellulose are added and blended in 1900 g distilled water. The resulting solution is sprayed onto the drilled and coated laminates in Hl-Coater pan coater. The lamina wall coated compartments are dried to yield the intermediate dosage form. The exterior release layer comprises 60 mg of verapamil HCl. Finally, a microporous membrane forming composition consisting of 50% cellulose acetate, 25% LUTROL® F127, and 25% LUTROL® F68 are dissolved in 99:1 acetone:water weight:weight at 5% solids is sprayed onto the drug coated trilayer dosage form using a Hl-COATER® pan coater until a coating thickness of 4 mils is applied. The manufacture of the drug form is completed by drying the system.

The dosage form provided by this example is indicated for twice a day (b.i.d.) therapy. The dosage form on entering the gastrointestinal tract delivers the first dose of 150 mg verapamil HCl after an initial drug-free interval, and several hours later commences delivery of the second dose of 60 mg verapamil HCl. The first dose provides antihypertensive action that reaches a therapeutic peak followed by a drug-free interval and then the second dose that provides antihypertensive action that reaches its therapeutic peak to enable the blood pressure to approach baseline values. The dosage form accordingly provides a favorable therapeutic index, with convenient, as a compliance-enhancing b.i.d. dosage form.

### DESCRIPTION OF METHOD OF PERFORMING THE INVENTION

A presently preferred embodiment of the invention pertains to a method for delaying the delivery of a drug to the gastrointestinal tract of a human followed by two separate deliveries of drug at controlled rates and continuously, which method comprises the steps of: (A) admitting orally into the human's gastrointestinal tract a dispensing device comprising:

(a) admitting the dosage form into the gastrointestinal tract of the warm-blooded animal, said dosage form comprising:

(1) a compartment;

(2) a semipermeable wall that surrounds and forms the compartment, said semipermeable wall comprising a composition that is permeable to the passage of fluid and is substantially impervious to the passage of a drug;

(3) an exterior drug layer comprising a dose amount of a first drug on the exterior surface of the semipermeable wall;

(4) microporous membrane coated around the exterior drug layer said microporous membrane comprising a composition that is permeable to the passage of fluid and is permeable to the passage of the first drug;

(5) a first composition in the compartment, said first composition being drug-free for producing a drug-free interval prior to the administration of drug from the compartment;

(6) a second composition in the compartment comprising a dose amount of a second drug for producing a therapeutic effect;

(7) a third composition in the compartment that expands in the presence of fluid that enters the device; and

(8) exit means in the semipermeable wall, external drug layer, and microporous membrane for connecting the exterior of the dosage form with the compartment;

(b) administering a dose of the first drug from the exterior drug layer by contacting the dosage form with gastrointestinal fluid;

(c) imbibing gastrointestinal fluid into the compartment thereby causing the third composition to expand and push against the second compartment; and

(d) administering the dose of the second drug from the drug composition after the first drug-free composition is released from the compartment.

Although only preferred embodiments of the invention are specifically disclosed and described above, it will be appreciated that many modifications and variations of the present invention are possible in light of the above teachings and within the purview of the appended claims without departing from the spirit and intended scope of the invention.

What is claimed is:

1. A once a day dosage form for delaying release of therapeutic agents comprising:
a) a three layer core including at least one drug layer;
b) a semipermeable membrane surrounding the core;
c) a drug coating at least partially surrounding the exterior of the semipermeable membrane;
d) a microporous membrane surrounding the exterior of the drug coating; and
e) an exit connecting the core to the exterior of the dosage form.

2. The dosage form of claim 1 wherein there is a delay in release of a drug layer subsequent to release of the drug coating.

3. The dosage form of claim 2 wherein there is a delay in release of the drug coating subsequent to administration of the dosage form to a subject.

4. The dosage form of claim 1 wherein there is a delay in release of the drug coating after administration to a subject.

5. The dosage form of claim 1 comprising an immediate release drug coating at least partially surrounding the exterior of the microporous membrane.

6. A dosage form for releasing doses of drug twice a day from a single dosage form, comprising:
a) a semipermeable membrane that surrounds and forms a compartment, the semipermeable membrane being permeable to the passage of fluid and substantially impermeable to the passage of a drug;
b) an exterior layer comprising a first drug on the exterior surface of the semipermeable membrane;
c) a microporous membrane coated on the surface of the exterior layer, the microporous membrane being permeable to the passage of fluid and permeable to the passage of the first drug;
d) a first composition in the compartment, being drug-free for producing a drug-free interval;
e) a second composition in the compartment comprising a dose amount of a second drug;
f) a third composition in the compartment that expands in the presence of fluid that enters the dosage form; and
g) an exit means in the semipermeable membrane, exterior layer, and microporous membrane for connecting the exterior of the dosage form with the compartment.

7. The dosage form of claim 6 comprising an immediate release drug coating at least partially surrounding the exterior of the microporous membrane.

8. The dosage form of claim 6 wherein the microporous membrane contains a pore-former.

9. The dosage form of claim 6 wherein at least one of the semipermeable membrane and the microporous membrane comprises cellulose acetate.

10. The dosage form of claim 6 wherein at least one of the semipermeable membrane and the microporous membrane comprises polymers selected from the group consisting of cellulose ester, cellulose diester, cellulose triester, cellulose ether, cellulose ester-ether, cellulose acrylate, cellulose diacrylate, cellulose triacrylate, methyl acrylate, poly(butyl methacrylate, 2-dimethylaminoethyl)methacrylate, methylmethacrylate) poly(methacrylic acid, methyl methacrylate)methyl ethyl acrylate copolymer, polyethyl (ethylacrylate, methyl methacrylate, trimethyl ammonioethyl methacrylate) and mixtures thereof.

11. The dosage form of claim 6 wherein exit means comprises at least one exit.

12. The dosage form of claim 6 wherein the exit means comprises a passage forming material that is removed from the means when the dosage form is in operation in the environment of use.

13. The dosage form of claim 6 wherein the first drug and the second drug are the same and are selected from the group consisting of verapamil, nimodipine, nitredipine, nisoldipine, nicardipine, felodipine, diltiazem, lidoflazine, tiapamil, guanabenz, isradipine, gallopamil, amlodipine, mioflazine, and caroverene.

14. The dosage form of claim 6 wherein the first drug and the second drug is selected from the group consisting of amyl nitrate, glyceryl trinitrate, octyl nitrite, sodium nitrite, erythrityl tetranitrate, isosorbide dinitrate, mannitol hexanitrate, pentaerythritol tetranitrate, pentritol, triethanolamine trinitrate, and trolnitrate phosphate.

15. A method for releasing doses of drug twice a day from a single dosage form to the gastrointestinal tract of a mammal, which method comprises:
a) admitting the dosage form into the gastrointestinal tract of the mammal, the dosage form comprising:
i) a compartment;
ii) a semipermeable membrane that surrounds and forms the compartment, the semipermeable membrane comprising a composition that is permeable to the passage of fluid and is substantially impervious to the passage of a drug;
iii) an exterior drug layer comprising a dose amount of a first drug on the exterior surface of the semipermeable membrane;
iv) a microporous membrane coated around the exterior drug layer, the microporous membrane comprising a composition that is permeable to the passage of fluid and permeable to the passage of the first drug;
v) a first composition in the compartment, the first composition being drug-free for producing a drug-free interval prior to the administration of drug from the compartment;
vi) a second composition in the compartment comprising a dose amount of a second drug for producing a therapeutic effect;
vii) a third composition in the compartment that expands in the presence of fluid that enters the device; and
viii) an exit means in the semipermeable wall, external drug layer, and microporous membrane for connecting the exterior of the dosage form with the compartment;
b) releasing a dose of the first drug from the exterior drug layer by contacting the dosage form with gastrointestinal fluid;
c) imbibing gastrointestinal fluid into the compartment thereby causing the third composition to expand and push against the second composition; and
d) releasing a dose of the second drug from the second composition after the first composition is released from the compartment.

16. The method of claim 15 wherein the first drug is administered following an initial drug-free interval of about 0 to about 2 hours.

17. The method of claim 15 wherein a drug-free interval is from about 1 to about 4 hours follows the administration of the dose of the first drug before the start of the administration of the dose of the second drug.

18. The method of claim 15 wherein at least one of the semipermeable membrane and the microporous membrane comprises a pore-former.

19. The method of claim 18 wherein at least one of the semipermeable membrane and the microporous membrane comprises cellulose acetate.

20. The method of claim 15 wherein at least one of the semipermeable wall and microporous membrane comprises polymers selected from the group consisting of:
cellulose ester, cellulose diester, cellulose triester, cellulose ether, cellulose ester-ether, cellulose acrylate, cellulose diacrylate, cellulose triacrylate, methyl acrylate, poly(butyl methacrylate, 2-dimethylaminoethyl)methacrylate, methylmethacrylate) poly(methacrylic acid, methyl methacrylate)methyl ethyl acrylate copolymer, polyethyl (ethylacrylate, methyl methacrylate, trimethyl ammonioethyl methacrylate) and mixtures thereof.

21. The method of claim 15 wherein exit means comprises at least one exit.

22. The method of claim 15 wherein the exit means comprises a passage forming material that is removed from the exit means when the dosage form is in contact with gastrointestinal fluid.

23. The method of claim 15 wherein the first drug and the second drug are the same and are selected from the group consisting of verapamil, nimodipine, nitredipine, nisoldipine, nicardipine, felodipine, diltiazem, lidoflazine, tiapamil, guanabenz, isradipine, gallopamil, amlodipine, mioflazine, and caroverene.

24. The method of claim 15 wherein the first drug and the second drug is selected from the group consisting of amyl nitrate, glyceryl trinitrate, octyl nitrite, sodium nitrite, erythrityl tetranitrate, isosorbide dinitrate, isosorbide mononitrate, mannitol hexanitrate, pentaerythritol tetranitrate, pentritol, triethanolamine trinitrate, and trolnitrate phosphate.

25. A method for releasing doses of drug three times a day from a single dosage form to the gastrointestinal tract of a mammal, which method comprises:
a) admitting the dosage form into the gastrointestinal tract of the mammal, the dosage form comprising:
i) a compartment;
ii) a semipermeable membrane that surrounds and forms the compartment, the semipermeable membrane comprising a composition that is permeable to the passage of fluid and is substantially impervious to the passage of a drug;
iii) an exterior drug layer comprising a dose amount of a first drug on the exterior surface of the semipermeable membrane;
iv) a microporous membrane coated around the exterior drug layer, the microporous membrane comprising a composition that is permeable to the passage of fluid and is permeable to the passage of the first drug;
v) an immediate release drug coating at least partially surrounding the exterior of the microporous membrane,
vi) a first composition in the compartment, the first composition being drug-free for producing a drug-free interval prior to the administration of drug from the compartment;
vii) a second composition in the compartment comprising a dose amount of a second drug for producing a therapeutic effect;
viii) a third composition in the compartment that expands in the presence of fluid that enters the device; and
ix) an exit means in the semipermeable membrane, external drug layer, and microporous membrane for connecting the exterior of the dosage form with the compartment;
b) releasing the immediate release drug coating by contacting the dosage form with the gastrointestinal fluid;
c) releasing the exterior drug layer after a first delay period from initial contact of the dosage form with gastrointestinal fluid;
d) imbibing gastrointestinal fluid into the compartment thereby causing the third composition to expand and push against the second composition; and
e) releasing the second drug composition after the first composition is released from the compartment.
